(19) United States
(12) Patent Application Publication
(10) Pub. No.: US 2004/0247628 A1
(43) Pub. Date: Dec. 9, 2004

(54) Kit for the preparation of a pharmaceutical composition

(76) Inventors: Frank-Christophe Lintz, Starnbery (DE); Manfred Keller, Bad Krozingen (DE)

Correspondence Address: Peter F Corless, Edwards & Angell, P O Box 55874, Boston, MA, 02205, US

(21) Appl. No.: 10/493,687

(22) PCT Filed: Oct. 24, 2002
(86) PCT No.: PCT/EP02/11918

(30) Foreign Application Priority Data
Oct. 24, 2001 (EP) 01 124 384.7

(51) Int. Cl. A61K 9/00

(52) U.S. Cl. 424/400

(57) ABSTRACT

The invention relates to pharmaceutical kits for the preparation of liquid compositions which can be administered to humans as aerosols for the diagnosis, prevention or treatment of human diseases. A kit according to the invention comprises a solid composition and a sterile aqueous liquid capable of dispersing or dissolving the solid composition to form a liquid composition which can be aerosolized. The solid composition of the kit comprises one or more active compounds and a water-soluble, low molecular weight excipient. Preferably, the solid composition comprises a sugar or a sugar alcohol, such as mannitol, lactose, or glucose.

### FIELD OF THE INVENTION

The present invention relates to pharmaceutical kits. More in particular, the invention relates to kits for the preparation of liquid compositions which can be administered to humans as aerosols. Such liquid compositions contain an active compound used for the diagnosis, prevention or treatment of human diseases which, for instance, affect the respiratory system.

### BACKGROUND OF THE INVENTION

The inhalation of aerosols has a long history in the treatment of various diseases and disorders. Today, a large number of pharmaceutical products for inhalation are marketed, most of which are used for the local therapy of the respiratory tract, while others administer a drug or diagnostic agent systemically.

Most commonly, pressurized metered dose inhalers (MDIs) are used to deliver bronchodilators and steroids for the treatment of asthma and other diseases of the respiratory system. Typically, MDIs contain liquified CFC propellants. Due to the negative ecological impact of CFC propellants, these MDIs are presently being replaced by devices containing alternative propellants, such as hydrofluoroalkanes, or by dry powder inhalers (DPIs). MDIs tend to be small and handy. Due to their convenience, they represented the vast majority of therapeutic inhalation devices in the past. However, apart from the ecological concerns associated with propellants, MDIs exhibit other pharmaceutical problems and disadvantages. For instance, they are quite inefficient in delivering a drug to the lung. Studies have shown that even when using an optimized MDI with an appropriate breathing technique, no more than about 15% of the actuated dose reaches the patient's lung. Holding chambers between the MDI and the mouth of the patient can improve the situation somewhat, but these devices are bulky and compromise the convenience of MDIs, so that they have not become widely accepted among patients. Another problem is that many patients have problems coordinating the actuation of the MDI with their breathing activity. This difficulty may be partly overcome by the use of the more recently introduced breath-actuated MDIs.

As an alternative to the pressurized, propellant-driven MDIs, dry powder inhalers (DPIs) have recently become increasingly popular. These devices do not contain a propellant. Instead, they rely on the patient's inspiration activity to disperse a powder formulation and to deliver it to the deep lung. A major disadvantage of most DPIs is that they require a substantial air flow, such as about 30 l/min, for effective pulmonary delivery. Many patients with impaired breathing function, such as asthmatic children or elderly people, are therefore not able to use DPIs.

Especially for these patients, nebulizers may be more useful to administer drugs via the pulmonary route. Pharmaceutical nebulizers produce inhalable aerosols from aqueous-based liquid formulations. Various types of nebulizers are used, with jet nebulizers presently being the most common type. The need for producing pressurized air makes jet nebulizers less handy, even though they are portable. On the other hand, they allow the patient to simply inhale an aerosol without requiring dose actuation.

Several pharmaceutical compounds are available as aqueous solutions for inhalation which can be aerosolized with a nebulizer. However, some drugs that are commonly used in the treatment of respiratory disporder are not available as aqueous liquid formulations because they are not sufficiently stable in water to allow for an acceptable shelf life. An example for such a compound is formoterol, or the salts of formoterol. In order to be able to deliver such water-labile compounds with a nebulizer, it would be desirable to provide a stable solid formulation of the compound which can be easily dispersed or dissolved and subsequently nebulized.

U.S. Pat. No. 6,014,970 discloses an aerosolizing system with a liquid dispenser and a cartridge containing a dry active ingredient. By actuating the liquid dispenser, a predetermined dose of liquid is transferred into the cartridge where it dissolves the drug. The drug solution is subsequently transferred to an aerosol generator that nebulizes it for inhalation.

DE 196 15 422 discloses a cartridge with a sealed chamber accomodating a solid formulation of an inhalable drug. The container itself holds a liquid, and the seals of the cartridge can be penetrated to dissolve the drug in the liquid. However, the device is specifially adapted for propellant-free metered dose inhalers and cannot easily be used with nebulizers.

U.S. Pat. No. 6,161,536 claims a pharmaceutical kit for aerosol administration of a drug with a nebulizer, the kit comprising a liquid and a solid component which are stored in individual water-impermeable containers. The solid component is an open matrix network comprising a drug and a pharmaceutically acceptable, water-soluble or water-dispersible carrier material. The liquid component is an aqueous vehicle that is provided in a sufficient quantity to dissolve the solid component within 15 seconds. However, some of the water-labile drugs whose aerosol administration is desirable may not easily be formulated as solid state open matrix networks. Furthermore, the carrier materials specifically disclosed in the document, i.e. gelatin, hydrolyzed gelatin, polyvinyl alcohol, polyvinylpyrrolidone and acacia are for physiological reasons not really recommendable for inhalation.

Thus there is a need for improved systems and pharmaceutical kits for preparing aerosolizable liquid compositions containing active compounds which have a low stability in aqueous solution.

It is therefore an object of the invention to provide a kit for preparing a liquid composition, the kit containing a water-sensitive active compound in a stabilized form. It is another object of the invention to provide a kit which is easy to handle, and which yields a liquid composition with improved tolerability when administered by inhalation.

### SUMMARY OF THE INVENTION

The invention provides a kit for preparing a liquid pharmaceutical composition for pulmonary administration, the kit comprising (a) a solid composition comprising an active compound and at least one pharmaceutically acceptable water-soluble excipient, said excipient having a molecular weight of no more than 1000 and a water solubility of at least 10 wt.-% at room temperature; and (b) a sterile aqueous liquid capable of dissolving the solid composition to form said liquid pharmaceutical composition. According to the invention, the active compound which can have a limited stability in aqueous solution is stabilized in its dry and solid form within the solid composition of the kit. Also within the kit, a sterile aqueous liquid is provided, which is capable of dissolving the solid composition to yield a liquid for pulmonary administration. The water-soluble, low molecular weight excipient primarily serves as a rapidly dispersible carrier for the drug, but it also contributes to the tolerability of the liquid for inhalation, e.g. by adjusting its osmolality to a physiological range. Preferred as such an excipient is a sugar or sugar alcohol.

The solid and the liquid compositions of the kit are stored in separate chambers within the same vessel or primary package. Alternatively, two or more different containers may be used to accommodate the two compositions of the kit. The composition may be designed as single-dose or multi-dose units. Multi-dose units may contain the sterile aqueous liquid within a metered dose dispenser.

The solid composition may represent a tablet, a lyophilized matrix, a powder, a lyophilized powder, granules, a film- or foil-shaped unit, or it may comprise a soluble or insoluble carrier which is coated with a soluble coating. In the latter case, the active compound is in the coating. For instance, glass or polymer beads can be used as carriers to provide a large surface area for the drug-containing coating material to aid its rapid dispersion. Typically, the solid composition is dissolved by the aqueous liquid provided in the kit within no more than about 30 seconds. To increase the dissolution rate, the kit may also contain an effervescent couple.

The invention is useful in the pulmonary delivery of active compounds for the diagnosis, prevention or treatment of diseases and conditions affecting the respiratory system, such as asthma, bronchitis, viral or bacterial infections, but also for the systemic delivery of drugs via the pulmonary route. A kit may contain one or more drugs which can be administered simultaneously. Further embodiments and useful applications of the invention are set forth below.

### DETAILED DESCRIPTION OF THE INVENTION

In a first aspect, the invention provides a kit for preparing a liquid pharmaceutical composition for pulmonary administration, the kit comprising (a) a solid composition comprising an active compound and at least one pharmaceutically acceptable water-soluble excipient, said excipient having a molecular weight of no more than 1000 and a water solubility of at least 10 wt.-% at room temperature; and (b) a sterile aqueous liquid capable of dissolving the solid composition to form said liquid pharmaceutical composition.

As used herein, a kit refers to a set of at least two compositions used for a specific purpose. In the present case, the purpose is the preparation of a liquid pharmaceutical composition for pulmonary administration. In most cases, such a liquid composition will resemble a solution, most preferably an aqueous solution. In some cases, however, the liquid may not be a solution in the strict physical sense, but rather a dispersion. As such, it may contain a dispersed colloidal material, suspended particles, dispersed liquid or semisolid droplets, liposomes, and the like.

For pulmonary administration, a liquid composition can be inhaled either through the nose or, more preferably, through the mouth. This is done, for instance, after nebulizing the liquid to form an aerosol, which is a dispersion of finely divided liquid droplets or solid particles in a gaseous phase. Various nebulizers are known and available for pharmaceutical applications. They make use of several methods of nebulization, such as air jet nebulization, ultrasonication, shear forces generated at multiple apertures (vibrating membrane technology), or electrohydrodynamic activation by an ionized electric field. The liquid itself can be prepared prior to its use from a solid composition and a liquid, both of which are provided with the kit.

The solid composition comprises the active compound which is to be administered. As used herein, an active compound refers to a substance or a mixture of closely related substances which is used for the diagnosis, prevention, or treatment of a disease. In this sense, the terms “drug” and “active compound” are interchangeable. In a preferred embodiment, the active compound is a drug used for the treatment of a disease or condition affecting the respiratory system, such as bronchitis, asthma, chronic obstructive pulmonary disease, allergies, cystic fibrosis, pneumonia, bronchiectasis, bronchiolitis, lung cancer and fibrosis, pulmonary hypertension, respiratory distress syndrome, bacterial or viral infections, tuberculosis and other diseases of the lower and upper respiratory tract, such as sinusitis. In another embodiment, drugs may be administered through the nose and/or lungs to reach the central circulation and to become systemically active. For instance, peptide or protein drugs, such as insulin, which are not bioavailable after oral administration, may be administered by inhalation to avoid injections. Examples of drugs that may be administered using the teachings of the invention include substances for diagnostic purposes such as metacholin or antiasthmatics, comprising beta-agonists, such as salbutamol, levalbuterol, formoterol, fenoterol, salmeterol, bambuterol, brocaterol, clenbuterol, terbutalin, tulobuterol, epinephrin, isoprenalin, orciprenalin, hexoprenalin; anticholinergics, such as tiotropium, oxitropium, ipratropium, glycopyrrolate; local anaesthetics, such as lidocain and derivatives thereof, mucolytics and surfactants, such as acetylcystein, ambroxol, carbocystein, tyloxapol, dipalmytoylphosphatidylcholin, recombinant surfactant proteins, D-nase; anti-inflammatory drugs comprising mediator cell inhibitors, such as cromoglycate, nedocromil, lidocaine, elastane-, leucotriene-, bradykinin-antagonists; corticosteroids, such as beclomethasone, betamethasone, budesonide, ciclesonide, flunisolide, fluticasone, icomethasone, mometasone, rofleponide, triamcinolone; bradykinine-, prostaglandine-, leucotriene- and platelet activating factor antagonists; antibiotics, including beta-lactam antibiotics, such as amoxicillin, piperacillin, clavulan acid, sulbactam; cephalosporines, e.g. cefaclor, cefazedon, Cefuroxim, Cefoxitin, cefodizim, cefsulodin, cefpodixim, cefixim; carbapenemes, such as imipenem and cilastatin; further monbactames, e.g aztrenonam; aminoglycosides, such as streptomycin, neomycin, colistin, paromomycin, kanamycin, gentamycin, amicacin, tobramycin, spectinomycine; tetracyclines, such as doxycyclin, minocycline; makrolides, such as erythromycine, clarithromycine, roxithromycine, azithromycine, josamycine, spiramycine; gyrase inhibitors or quinolones, such as ciprofloxacin, ofloxacine, levofloxacine, pefloxacine, lomefloxacine, fleroxacine, clinafloxacine, sitafloxacine, gemifloxacine, balofloxacine, trovafloxacine, gatifloxacine, moxifloxacine; sulfonamides and nitroimidazoles, including metronidazol, timidazol, chloramphenicol, lincomycine, clindamycine, fosfomycine; glycopeptides such as vancomycine, teicoplanine; peptide antibiotics, such as peptide 4; tuberculostatics, e.g. rifampicine, isoniacide, cycloserine, terizidone, ansamycine; antimycotics and antifungals, such as clotrimazol, oxiconazol, miconazol, ketoconazol, itraconazol, fluconazol; polyene antibiotics, such as amphotericine B, natamycine, nystatine, colistine, flucytosine; chemotherapeutics like pentamidine; immunesuppressors and immunemodulators, cytokines, dimepranol-4-acetate amideo benzoate, thymopentin, interferones, filgrastine, interleukine, azathioprine, ciclosporine, tacrolimus, sirolimus, rapamycine; drugs to treat pulmonary hypertension, such as prostacycline analogs, iloprost, remodulin, phosphodiesterase inhibitors, such as sildenafil, vardenafil, endothelian receptor antagonists, such as bosentane, virustatics, including podophyllotoxine, vidarabine, tromantadine, zidovudine; proteinase inhibitors, such as a-anti-trypsin; antioxidants, such as tocopherols, glutathion; pituitary hormones, hypothalamic hormones, regulatory peptides and their inhibiting agents, corticotropine, tetracosactide, choriogonandotropine, urofolitropine, urogonadotropine, saomatotropine, metergoline, desmopressine, oxytocine, argipressine, ornipressine, leuproreline, triptoreline, gonadoreline, busereline, nafareline, goselerine, somatostatine; parathyroide gland hormones, calcium metabolism regulators, dihydrotachysterole, calcitonine, clodronic acid, etidronic acid; thyroid gland therapeutics; sex hormones and their inhibiting agents, anabolics, androgens, estrogens, gestagenes, antiestrogenes; cytostatics and metastasis inhibitors, alkylants, such as nimustine, melphanlane, carmustine, lomustine, cyclophosphosphamide, ifosfamide, trofosfamide, chlorambucil, busulfane, treosulfane, prednimustine, thiotepa; antimetabolites, e.g. cytarabine, fluorouracil, methotrexate, mercaptopurine, tioguanine; alkaloids, such as vinblastine, vincristine, vindesine; antibiotics, such as alcarubicine, bleomycine, dactinomycine, daunorubicine, doxorubicine, epirubicine, idarubicine, mitomycine, plicamycine; complexes of secondary group elements (e.g. Ti, Zr, V, Nb, Ta, Mo, W. Pt) such as carboplatinum, cis-platinum and metallocene compounds such as titanocendichloride; amsacrine, dacarbazine, estramustine, etoposide, beraprost, hydroxycarbamide, mitoxanthrone, procarbazine, temiposide; anti-migraine drugs, such as proxibarbal, lisuride, methysergide, dihydroergotamine, ergotamine, clonidine, pizotifene; hypnotics, sedatives, benzodiazepines, barbiturates, cyclopyrrolones, imidazopyridines, antiepileptics, barbiturates, phenyloin, primidone, mesuximide, ethosuximide, sultiam, carbamazepin, valproic acid, vigabatrine; antiparkinson drugs, such as levodopa, carbidopa, benserazide, selegiline, bromocriptine, amantadine, tiapride; antiemetics, such as thiethylperazine, bromopride, dompeddone, granisetrone, ondasetrone, tropisetrone, pyridoxine; analgesics, such as buprenorphine, fentanyl, morphine, codeine, hydromorphone, methadone, fenpipramide, fentanyl, piritramide, pentazocine, buprenorphine, nalbuphine, tilidine; drugs for narcosis, such as N-methylated barbiturates, thiobarbiturates, ketamine, etomidate, propofol, benzodiazepines, droperidol, haloperidol, alfentanyl, sulfentanyl; antirheumatism drugs including tumor necrosis factor-alfa, nonsteroidal antiinflammatory drugs; antidiabetic drugs, such as insulin, sulfonylurea derivatives, biguanids, glitizols, glucagon, diazoxid; cytokines, such as interleukines, interferones, tumor necrosis factor (TNF), colony stimulating factors (GM-CSF, G-CSF, M-CSF); proteins, e.g. epoetine, and peptides, e.g. parathyrin, somatomedin C; heparine, heparinoids, urokinases, streptokinases, ATP-ase, prostacycline, sexual stimulants, or genetic material. Among the more preferred active compounds are albuterol, salbutamol, R-salbutamol, bitolterol, carbuterol, tretoquinol, formoterol, clenbuterol, reproterol, pirbuterol, tulobuterol, procaterol, bambuterol, mabuterol, tiaramide, budenoside, fluticasone, beclometasone, deflazacort, TBI-PAB, flunisolide, cloprednol, emedastine, epinastine, oxatomide, azelastine, pemirolast, repirinast, suplatast, nedocromil, oxitropium, flutropium, triamcinolone, allergy vaccines, zafirlukast, montelukast, ramatroban, seratrodast, TJ-96, ibudilast, tranilast, Iodoxamide, TO-194, pranlukast, letosteine, ketotifen, amlexanox, zileuton, Efamol Marine, tazanolast, ribavirin, pentamidine, colistin, amphotericin B, ozagrel, including their derivatives, salts, conjugates, isomers, epimers, diastereomers, or racemic mixtures.

The invention is particularly useful for the administration of compounds that are not sufficiently stable in an aqueous liquid to allow for a shelf life of more than about 2 years without refrigeration. Even more preferred is the kit of the invention in which the active compound is stable in water for no longer than about 1 year at room temperature. In a still more preferred embodiment, the active compound is not stable in water for more than about 6 months. As used herein, the stability of a compound in water means that at least 90 wt-% of the compound remain chemically unchanged after the designated period of time.

In addition to the active compound, the solid composition comprises a pharmaceutically acceptable, water-soluble excipient with a molecular weight of not more than approximately 1,000 and a solubility in water of at least about 10 wt.-%, as measured at room temperature. The excipient thus defined is useful in several aspects. First, it serves as a pharmacologically substantially inert carrier for the active compound, as in many case the drug itself does not have the physicochemical properties that would allow it to be formulated without a carrier substance. For instance, some drugs are administered in such small doses that, without a carrier, they would be difficult to handle or dose precisely. In other cases, the drug by itself would not dissolve at an acceptable rate without a hydrophilic excipient. Thus, the invention calls for an excipient which is water-soluble as defined in claim 1. More preferably, the excipient has a solubility of at least about 20 wt.-% in water, thus representing a highly soluble molecule. In another embodiment, the excipient has a molecular weight of less than 500.

Useful excipients according to the invention are, for example, mono-, di- and oligosaccharides, sugar alcohols, organic or inorganic salts, organic or inorganic acids, or amino acids. Particularly preferred are mannitol, lactose, glucose, isomalt, sucrose, and trehalose, especially mannitol and lactose. The compounds have an excellent tolerability after pulmonary administration, and can be pharmaceutically processed as carriers in many ways. Due to their nature as low molecular weight compounds they also exhibit substantial osmotic activity, for which reason they are useful excipients for adjusting the tonicity of the final liquid composition to be administered, which further contributes to the tolerability of that liquid composition to the lung.

Furthermore, the low molecular weight excipient has the advantage over the polymers suggested as carriers in prior art that it will be eliminated faster from the lungs, while polymers tend have a longer residence time leading to their accumulation after frequent dosings. In one of the preferred embodiments of the invention, the solid composition is therefore substantially free of polymers. However, if polymeric excipients cannot be avoided altogether, they should preferably be used with care, i.e. in relatively small amounts, for instance not exceeding a concentration of about 50 wt.-% in the solid composition of the kit, or they should be polymers with relatively low molecular weight, which are also eliminated from the lungs at an acceptable rate. In another embodiment, the solid composition is therefore substantially free of polymers with a molecular weight of more than 10,000.

Preferably, the water-soluble excipient is present in the solid composition of the kit at a concentration of not less than about 10 wt.-%, and due to its tolerability it may also be incorporated in high concentrations when needed, such as up to 99.5 wt.-%. In most cases, the concentration will range from about 20 wt.-% to about 99 wt.-%, depending on the unit dose and on the physicochemical properties of the drug. Highly potent drugs, such as formoterol, may require a relatively large concentration of excipient, such as 80 wt.-% to 99.5 wt.-%.

If the water-soluble excipient is an organic or inorganic acid, or an organic or inorganic salt, or an amino acid, it may serve an additional function within the solid composition, and especially in the final liquid composition, which is to adjust the pH to a value at which the active compound is relatively stable, and to further increase the tolerability of the aerosol to the lungs. Such a tolerable pH is frequently called isohydric, i.e. the pH of the solution approximately equals the pH of the environment at the site of administration, such as the mucus layer covering the respiratory tract; or it may be termed euhydric when the pH does not match the physiological pH, but is adjusted to a value which still is well tolerated by the organism. Compounds useful as water-soluble excipients which also affect the pH include e.g. citric acid, tartaric acid, sodium dihydrogen phosphate, disodium dihydrogen pyrophosphate.

To achieve the desired effects, it may be useful to incorporate more than one water-soluble low molecular weight excipient into the solid composition. For instance, one excipient as defined in claim 1 may be selected for its drug carrier and diluent capability, while another excipient may be selected to adjust the pH. If the final liquid composition needs to be buffered, two excipients which together form a buffer system may be selected.

The solid composition may also comprise further substances and ingredients which may or may not be water-soluble, and whose molecular weight may optionally exceed 1,000. For instance, it may comprise a surfactant to increase the wettability of the active compound or to improve the dissemination of the aerosol droplets in the lungs. A surfactant should also be pharmaceutically acceptable in the amount that is incorporated in the formulation. Examples of surfactants that may be used are phospholipids, Pluronics, Tweens, and tyloxapol. The most preferred surfactants are Tween 80 and tyloxapol.

Referring to the liquid that is provided in the kit, several basic requirements can be defined. According to the invention, the liquid is an aqueous liquid, which is herein defined as a liquid whose major component is water. The liquid does not necessarily consist of water only; however, in one of the preferred embodiments it is indeed purified water. In another embodiment, the liquid contains other components or substances, preferably other liquid components, but possibly also dissolved solids. Liquid components other than water which may be useful include propylene glycol, glycerol, and polyethylene glycol. One of the reasons to incorporate a solid compound as a solute is that such a compound is needed or desirable in the final liquid composition, but is incompatible with the solid composition or with a component thereof, such as the active ingredient.

Another requirement for the liquid supplied with the kit is that it is sterile. As an aqueous liquid, it would be subject to the risk of considerable microbiological contamination and growth if no measures were taken to ensure sterility. In order to provide a substantially sterile liquid, it is either necessary to incorporate an effective amount of an acceptable antimicrobial agent or preservative, or to sterilize the liquid prior to providing it and to seal it with an air-tight seal. Preferably, the liquid is a sterilized liquid free of preservatives and provided in an appropriate air-tight container. However, according to another embodiment in which the kit contains multiple doses of the active compound, the liquid may be supplied in a multiple-dose container, such as a metered-dose dispenser, and may require a preservative to prevent microbial contamination after the first use.

A further requirement is that the sterile aqueous liquid is capable of dissolving the solid composition of the kit to form a liquid composition which can be aerosolized and inhaled. Such capability is, among other factors, a function of the selected amount and, potentially, the composition of the liquid. To allow easy handling and reproducible dosing, the sterile aqueous liquid should be able to dissolve the solid composition within a short period of time, possibly under gentle shaking. Preferably, the final liquid should be ready to use after no longer than about 30 seconds. More preferably, the solid composition is dissolved within about 20 seconds, and still more preferably within about 10 seconds. As used herein, the terms “dissolve(d)”, “dissolving”, and “dissolution” refer to the disintegration of the solid composition and the release, i.e. the dissolution, of the active compound. As a result of dissolving the solid composition with the sterile aqueous liquid a liquid composition is formed in which the active compound is contained in the dissolved state. As used herein, the active compound is in the dissolved state when at least about 90 wt.-% are dissolved, and more preferably when at least about 95 wt.-% are dissolved. To measure disintegration and/or dissolution times, standard pharmacopoeial methods may be used. However, the methods must be selected to be appropriate for the specific form in which the solid composition of the kit is supplied. For instance, if the solid composition is a powder, it may be meaningless to measure disintegration. In other cases, an official method for measuring the dissolution time of the drug may not be relevant to the actual use of the kit. In these cases, it may be better to determine the dissolution time under conditions which resemble those achieved by following the instructions for preparing the final liquid composition given in the kit.

With regard to the basic kit design, it primarily depends on the specific application whether it is more useful to accommodate the aqueous liquid and the solid composition within separate chambers of the same container or primary package, or whether they should be provided in separate containers. If separate containers are used, these are provided as a set within the same secondary package. The use of separate containers is especially preferred for kits containing two or more doses of the active compound. There is no limit to the total number of containers provided in a multi-dose kit. In one of the preferred embodiments for multiple-dose kits, the solid composition is provided as unit doses within multiple containers or within multiple chambers of a container, whereas the aqueous liquid is provided within one chamber or container. In this case, a favorable kit design provides the liquid in a metered-dose dispenser, which may consist of a glass or plastic bottle closed with a dispensing device, such as a mechanical pump for metering the liquid. For instance, one actuation of the pumping mechanism may dispense the exact amount of liquid for dissolving one dose unit of the solid composition.

In another preferred embodiment for multiple-dose kits, both the solid composition and the aqueous liquid are provided as matched unit doses within multiple containers or within multiple chambers of a container. For instance, two-chambered containers can be used to hold one unit of the solid composition in one of the chambers and one unit of liquid in the other. As used herein, one unit is defined by the amount of drug present in the solid composition, which is one unit dose. Such two-chambered containers may, however, also be used advantageously for kits containing only one single drug dose.

In a preferred embodiment, a blister pack having two blisters is used, the blisters representing the chambers for containing the solid composition and the sterile aqueous liquid in matched quantities for preparing a dose unit of the final liquid composition. As used herein, a blister pack represents a thermoformed or pressure-formed primary packaging unit, most likely comprising a polymeric packaging material that optionally includes a metal foil, such as aluminum. The blister pack may be shaped to allow easy dispensing of the contents. For instance, one side of the pack may be tapered or have a tapered portion or region through which the content is dispensable into another vessel upon opening the blister pack at the tapered end. The tapered end may represent a tip. An simplified example of such a two-chamber blister pack is illustrated in FIG. 1.

More preferably, the two chambers of the blister pack are connected by a channel, the channel being adapted to direct fluid from the blister containing the sterile aqueous liquid to the blister containing the solid composition. During storage, the channel is closed with a seal. In this sense, a seal is any structure that prevents the aqueous liquid from contacting the solid composition. The seal is preferably breakable or removable; breaking or removing the seal when the kit is to be used will allow the aqueous liquid to enter the other chamber and dissolve the solid composition. The dissolution process may be improved by shaking the blister pack. Thus, the final liquid composition for inhalation is obtained, the liquid being present in one or both of the chambers of the pack connected by the channel, depending on how the pack is held.

According to another preference, one of the chambers, preferably the one which is closer to the tapered portion of the blister pack, communicates with a second channel, said channel extending from the chamber to a distal position of the tapered portion. During storage, this second channel does not communicate with the outside of the pack but is closed in an air-tight fashion. Optionally, the distal end of the second channel is closed by a breakable or removable cap or closure, which may e.g. be a twist-off cap, a break-off cap, or a cut-off cap.

The solid composition itself can be provided in various different types of dosage forms, depending on the specific application of the kit, the physicochemical properties of the drug, the desired dissolution rate, cost considerations, and other criteria. In one of the embodiments, the solid composition is a single unit. This implies that one unit dose of the drug is comprised in a single, physically shaped solid form or article. In other words, the solid composition is coherent, which is in contrast to a multiple unit dosage form, in which the units are incoherent.

Examples of single units which may be used as dosage forms for the solid composition include tablets, such as compressed tablets, film-like units, foil-like units, wafers, lyophilized matrix units, and the like. In a preferred embodiment, the solid composition is a highly porous lyophilized form. Such lyophilizates, sometimes also called wafers or lyophilized tablets, are particularly useful for their rapid disintegration, which also enables the rapid dissolution of the active compound.

On the other hand, for some applications the solid composition may also be formed as a multiple unit dosage form as defined above. Examples of multiple units are powders, granules, microparticles, pellets, beads, lyophilized powders, and the like. In one of the preferred embodiments, the solid composition is a lyophilized powder. Such a dispersed lyophilized system comprises a multitude of powder particles, and due to the lyophilization process used in the formation of the powder, each particle has an irregular, porous microstructure through which the powder is capable of absorbing water very rapidly, resulting in quick dissolution.

Another type of multiparticulate system which is also capable of achieving rapid drug dissolution is that of powders, granules, or pellets from water-soluble excipients which are coated with the drug, so that the drug is located at the outer surface of the individual particles. In this type of system, the water-soluble low molecular weight excipient as defined in claim 1 is useful for preparing the cores of such coated particles, which can be subsequently coated with a coating composition comprising the drug and, preferably, one or more additional excipients, such as a binder, a pore former, a saccharide, a sugar alcohol, a film-forming polymer, a plasticizer, or other excipients used in pharmaceutical coating compositions.

In another preferred embodiment, the solid composition of the kit resembles a coating layer which is coated on multiple units made of insoluble material. Examples of insoluble units include beads made of glass, polymers, metals, and mineral salts. Again, the desired effect is primarily rapid disintegration of the coating layer and quick drug dissolution, which is achieved by providing the solid composition in a physical form that has a particularly high surface-to-volume ratio. Typically, the coating composition will, in addition to the drug and the water-soluble low molecular weight excipient, comprise one or more further excipients, such as those mentioned above for coating soluble particles, or any other excipient known to be useful in pharmaceutical coating compositions.

According to the invention, it is further preferred that the solid composition and the sterile aqueous liquid are formulated and adapted to each other to yield upon their combination a liquid composition that is eutonic or isotonic, which feature improves the tolerability of the aerosol to the lung. As used herein, a eutonic liquid is one that has an osmotic pressure which is in the same broad range as the physiological fluids of the body. More specifically, the liquid composition has an osmolality in the range from about 150 mOsmol/kg to about 500 mOsmol/kg, and more preferably from about 200 mOsmol/kg to about 450 mOsmol/kg. In another embodiment, the final aerosol composition has an osmolality from about 250 mOsmol/kg to about 400 mOsmol/kg. The osmolality is achieved, for instance, by selecting the appropriate amounts of the water-soluble low molecular weight excipients, taking into consideration the type and amount of compounds which are also present in both the solid composition and the aqueous liquid.

In a further embodiment, the solid composition and the sterile aqueous liquid are formulated and adapted to each other to yield upon their combination a liquid composition for inhalation that is euhydric or even isohydric. As used herein, euhydric refers to the pH of the liquid composition, which is within a substantially tolerable range, while isohydric refers to a pH that is substantially similar to that of physiological fluids. Preferably, the inhalable liquid composition obtained by dissolving the solid composition with the aqueous liquid will have a pH within the range of about 3.5 to about 10.5. More preferably, the pH will be in the range of about 4.5 to about 9.5, which is even more tolerable to the lungs. Highly preferred are pH values that are still closer to the isohydric pH, such as from about 5.5 to about 8.5 or from about 6.0 to about 8.0.

As another option for further improving the dissolution behavior of the solid composition, an effervescent couple may be incorporated into the two kit component from which the inhalable liquid composition is prepared. An effervescent couple comprises two or more substances which are capable of reacting with each other to form a gas. In most pharmaceutical applications, the gas is carbon dioxide, which can be safely generated from substances that are physiologically acceptable. Typically, an effervescent couple comprises a basic compound, such as a basic salt, which is capable of releasing carbon dioxide, and an acid or an acidic salt to react with the basic salt in the presence of water. Examples for useful basic salts capable of releasing carbon dioxide are sodium carbonate, sodium bicarbonate, potassium carbonate, potassium bicarbonate, sodium glycine cabonate, and calcium carbonate. Examples for acceptable acids and acidic salts include citric acid, ascorbic acid, hydrochloric acid, phosphoric acid, sulfuric acid, glutamic acid, aspartic acid, and the like.

The effervescent couple is preferably stabilized in the kit by incorporating one member into the solid composition and the other member into the sterile aqueous liquid. If the effervescent couple comprises more than one acidic or more than one basic compound, the aqueous liquid may contain either any or all acidic compounds or any or all basic compounds of the couple.

### BRIEF DESCRIPTION OF THE DRAWINGS

FIG. 1 represents a schematic of an example of a dual-chamber blister pack for a kit according to the invention. The kit (1) formed of primary packaging material (2) is shaped to have a tapered end (3). It comprises a first compartment or chamber (4) containing a solid composition (6), and a second compartment (5) containing a sterile aqueous liquid (7). The compartments are connected by a channel (8) which is closed with a breakable seal (9). A second breakable seal shaped as a twist-off cap (10) seals a second channel (11) extending from the second chamber (5) through the tapered end (3) from the outside.

Alternatively, the kit can be constructed in a way, that blister made out of water impermeable materials such as PVDC sealed with an aluminium foil is fitted to a vial containing the liquid. The aluminium foil is used as seal to close the vial by means of a centrally opened screw holding the blister and to tighten the entire kit system. The thermoplastic part of the blister being for instance dome shaped can pressed by the thumb and will perforate by means of a plastic ring in the blister for instance in a shape of a Mercedes star the aluminium foil. The liquid and the poweder can be mixed by shaking and by removing the screw cap with the blister, the resulting product can be transferred in the inhalation device for administration of the drug into the nose or lungs.

FIG. 2 shows the particle size distribution and mean diameter of the budesonide suspension of example 3 before spray-drying (left) and after spray-drying with subsequent redispersion (right).

The invention will be further understood by reference to the following, nonlimiting examples.

### EXAMPLE 1

An aqueous solution containing 5.2% mannitol, 8 μg/ml formoterol fumarate and 0.1% polysorbate 80 was prepared using standard laboratory equipment and overnight stirring. No heating was applied. Sterile glass lyophilization vials were each filled with 2 mL of the solution using a sterile graduated pipette after filtration through a 0.22 μm cellulose filter for particle removal and sterility. All processing steps were done in a laminar air flow box.

The solution was lyophilized according to the conditions listed in table 1.

TABLE 1
Lyophilization conditions

| Step | Time (h) | Temperature (° C.) | Pressure (mbar) |
|---|---|---|---|
| Freezing | 6 | −40 | 1013 |
| Primary drying | 18 | −10 | 0.250 |
| Secondary drying | 18 | +20 | 0.04 |

The lyophilizates thus obtained were visually acceptable, with a volume of approx. 2 cm³.

The lyophilizates were capable of dissolving upon addition of 1 mL of sterile purified water. The resulting solution was sterile and isotonic (approx. 380 mOsmol/kg). Due to the presence of a surfactant, the dissolution time is relatively short (approx. 1 min), even without shaking the vial during dissolution. In order to further reduce dissolution times of the lyophilizates, the amount of surfactant was increased as shown in table 2.

TABLE 2
Influence of surfactant concentration on dissolution time

| Surfactant (%) | Dissolution time (sec) |
|---|---|
| 0.1 | 73 |
| 0.2 | 40 |
| 0.5 | 30 |

All reconstituted solutions could be nebulized by means of jet nebulizers (e.g. PARI LC PLUS®) or a vibrating membrane type nebulizers (e.g. PARI e-FLOW™).

### EXAMPLE 2

A powder mixture containing 50.0 mg of formoterol fumarate and 450.0 mg of mannitol was prepared using a standard laboratory blender in a stainless steel mixing vessel. In a second step, an aqueous solution was prepared according to the following composition:

| | | |
|---|---|---|
| Powder mixture | 0.05 | g |
| Mannitol | 21.62 | g |
| Polysorbate 80 | 0.21 | g |
| Purified water | ad 875.25 | g |

After filtration through a 0.22 μm cellulose filter for particle removal and sterility, aliquots of 2.1 mL of the solution were transferred into sterile glass vials using a sterile graduated glass pipette. The solution was freeze dried according to the following conditions:

| | |
|---|---|
| Freezing: | 4 hours (−40° C., 1013 mbar) |
| Primary drying: | 18 hours (−10° C., 0.25 mbar) |
| Secondary drying: | 18 hours (+20° C., 0.04 mbar). |

The resulting product was a white free flowing powder. Upon addition of 1 mL of water for injection through the vial cap using a pre-filled syringe, the powder re-dissolved in approx. 2 seconds without shaking. The resulting solution was isotonic, sterile and ready for nebulization with jet nebulizers (e.g. PARI LC PLUS®) or vibrating membrane type nebulizers (e.g. PARI e-Flow™).

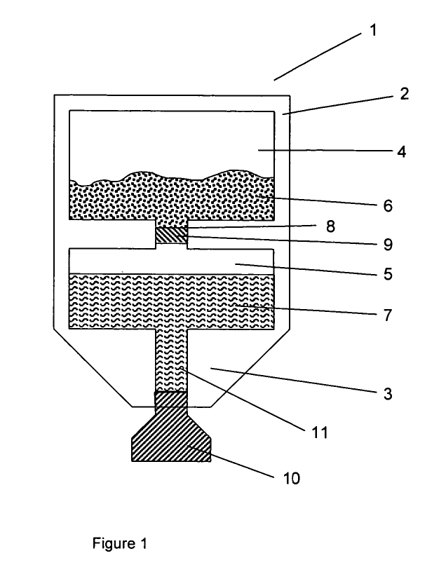

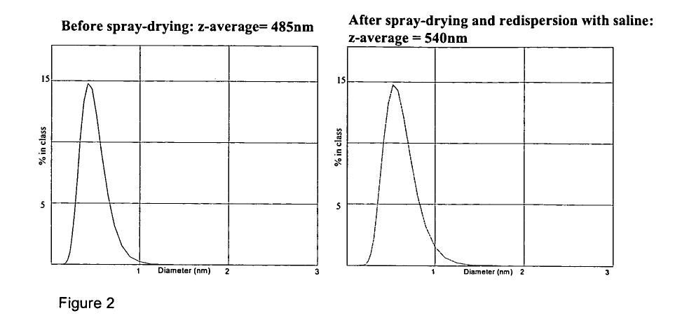

Alternatively, the powder can be transferred into one of the blisters of a sterile dual blister pack containing in a second cavity a sterile liquid with or without drug as re-dispersion solvent (see FIG. 1). Upon re-dissolution, the solution is poured into the nebulizer by means of a tip in the blister.

### EXAMPLE 3

A 0.5% aqueous Tween 80® solution is prepared using standard laboratory equipment without heating. 1.0% Budesonide is added under gentle stirring. This slurry is pre-homogenized using an Ultra Turrax® mixer (11,000 rpm, 1 min). The resulting suspension is homogenized by means of high-pressure homogenization, using an Microfluidics M110-EH equiped with Z- and Y-chambers under active cooling. Homogenization conditions are: 1,500 bar, 50 cycles.

The resulting submicron suspension, with particle sizes below 1 μm is spray dried using a Büchi spray-dryer equiped with a standard two-channel nozzle at an inlet air temperature of about 70° C. The obtained white and free-flowing powder with a particle size of about 5 μm is transferred into one compartment of a blister pack. The appropriate amount of sterile saline as re-dispersion agent (0.9% NaCl) is packed in the second compartment.

Upon mixing the two compounds within the blister, a sterile and isotonic suspension is obtained, with particle sizes ranging below 1 μm (see FIG. 2). This suspension is ready for nebulization by means of jet nebulizers (e.g. PARI LC PLUS®) or vibrating membrane type nebulizers (e.g. PARI e-Flow™).

### EXAMPLE 4

An aqueous solution containing 3% mannitol, 10% aztreonam-disodium and 0.01% tyloxapol was prepared using standard laboratory equipment. 2 mL of the solution were transferred into sterile glass lyophilization vials using a sterile graduated pipette after filtration through a 0.22 μm cellulose filter for particle removal and sterility. All processing steps were done in a laminar air flow box. The lyophilization process was carried out as described in example 1. The resulting lyophilizate was dissolved in 2 ml water and can be used for nebulization by means of jet nebulizers (e.g. PARI LC PLUS®) or vibrating membrane type nebulizers (e.g. PARI e-Flow™).

### EXAMPLE 5

An aqueous solution containing 1% mannitol, 0.003% formoterol fumarate and 0.001% tyloxapol was prepared using standard laboratory equipment. After filtration through a 0.22 μm cellulose filter, 0.5 mL were transferred into one cavity of a sterile dual chamber blister using a sterile graduated pipette. All processing steps were done in a laminar air flow box. The lyophilization process was carried out as described in example 1. Thereafter, 0.5 ml of a sterile solution containing 0.5% oxitropiumbromide and sodium chloride each was filled in the second cavity of the dual chamber blister. The dual chamber blister was than seal by a PVC-coated aluminium folie. Prior to nebulization, the liquids were mixed by perforation of the separation membrane due to pressure on one cavity allowing the liquids to mix. After pressing the liquids 3 times forth and back, the content was transferred into a nebulizer for administration of the aerosol into the lungs.

### EXAMPLE 6

An aqueous solution containing 0.1% mannitol, 0.005% formoterol-fumarate and 0.001% tyloxapol was prepared using standard laboratory equipment. After filtration through an a 0.22 μm cellulose filter, 0.25 mL were transferred into cavity no. 1 of a sterile dual chamber blister, respectively. All processing steps were done in a laminar air flow box. The lyophilization process was carried out as described in example 1.

40 mg of a spray dried submicron suspension—prepared as described in example 3—containing fluticasone-propionate and mannitol (1 mg/50 mg) was added in a laminar box to the formoterol lyophilizate. Thereafter, a 0.5 ml of a sterile solution containing 0.3% tiotropiumbromide bromide was filled in the second cavity of the dual chamber blister. The dual chamber blister was than seal by a PVDC-coated aluminium folie. All processing steps were done in a laminar air flow box.

Prior to nebulization, the powder mixture was dissolved by perforation of the separation membrane due to pressure on the cavity containing the liquid, allowing the liquid to penetrate into the second cavity to dissolve and disperse the powder mixture. After pressing the blister with the dissolved/dispersed powder in the liquid 3 times forth and back, the content was transferred into a nebulizer for immediate nebulization.

### EXAMPLE 7

An aqueous mixture containing 2.5% mannitol, 10% aztreonam-lysinate and 0.025% polysorbate 80 was prepared using standard laboratory equipment. 1 mL of the mixture was transferred into sterile glass lyophilization vials using a sterile graduated pipette. All processing steps were done in a laminar air flow box. The lyophilization process was carried out as described in example 1. The resulting lyophilizate was sterilized by qamma irradiation. Prior to use, the lyophilizate was dissolved by vigorously shaking in 2 ml sodium hydrogen carbonate solution and transferred into a nebulizer for immediate aerosolization.

### EXAMPLE 8

An aqueous solution containing 1.0% sildenafil citrate dissolved in a mixture consisting of 2% mannitol, 1% tyloxapol and 0.17% sodium chloride was prepared using standard laboratory equipment. 1 ml of the solution was filtered through a 0.22 μm cellulose filter into a sterile vial with a centrally open screw cap for holding a PVDC-blister sealed with an aluminium foil. 50 mg of a sterile spray-dried bosentane nanosuspension was transferred under aseptic conditions into a PVDC-blister containing a sterile glass sphere for easier penetration of the aluminium foil. By means of a pressure on the rounded part of the PVDC-blister, the aluminium foil was perforated with the help of the inserted glass sphere and allowed to disperse by vigorous shaking the powdered bosentane nanosuspension with the sildenafil-citrate solution. The homogenous dispersion was transferred into a nebulizer for administration of the aerosol into the lungs.

1-36. (cancelled)

37. A kit for preparing a liquid pharmaceutical composition for pulmonary administration, the kit comprising:
(a) a solid composition comprising an active compound and at least one pharmaceutically acceptable water-soluble excipient, said excipient having a molecular weight of no more than 1000 and a water solubility of at least 10 wt.-% at room temperature;
(b) a sterile aqueous liquid capable of dissolving the solid composition to form said liquid pharmaceutical composition.

38. The kit of claim 37, wherein the water-soluble excipient has a molecular weight of less than 500 and/or a water solubility of at least 20 wt.-% at room temperature.

39. The kit of claim 38, wherein the water-soluble excipient is selected from the group consisting of mono- and disaccharides, sugar alcohols, organic or inorganic salts, organic or inorganic acids, and amino acids.

40. The kit of claim 39, wherein the water-soluble excipient is selected from the group consisting of mannitol, lactose, and glucose.

41. The kit of claim 37, wherein the concentration of the water-soluble excipient in the solid composition is from about 10 wt.-% to about 99.5 wt.-%.

42. The kit of claim 37, wherein the solid composition comprises at least two pharmaceutically acceptable water-soluble excipients having a molecular weight of no more than 1000 and a water solubility of at least 10 wt.-% at room temperature.

43. The kit of claim 37, wherein the solid composition and the sterile aqueous liquid are accommodated in separate containers.

44. The kit of claim 43, comprising multiple doses of the active compound.

45. The kit of claim 44, wherein the sterile aqueous liquid is contained in a metered dose dispenser.

46. The kit of claim 37, wherein the solid composition and the sterile aqueous liquid are accommodated in separate chambers of the same container.

47. The kit of claim 46, comprising one single dose of the active compound.

48. The kit of claim 47, comprising a blister pack having a narrowed portion forming a tip, said pack comprising:
(a) a first blister chamber containing a solid composition, said composition comprising an active compound and at least one pharmaceutically acceptable water-soluble excipient;
(b) a second blister chamber containing a sterile aqueous liquid capable of dissolving said solid composition to form a liquid composition for nasal or pulmonary administration;
(c) a first channel extending from the first to the second blister chamber, said first channel being closed with a breakable or removable seal; and
(d) a second channel extending from the first or the second blister chamber to a distal position of the tip,
wherein the contents of the first and second blister chambers can be mixed by perforation of the connecting seals of both chambers by means of external pressure and wherein one of the blister chambers may contain a glass sphere, ring or any aid facilitating the perforation of the seal.

49. The kit of claim 48, further comprising a breakable or removable closure positioned at the distal end of the second channel.

50. The kit of claim 37, wherein the solid composition is substantially free of polymers.

51. The kit of claim 37, wherein the solid composition further comprises a surfactant.

52. The kit of claim 51, wherein the surfactant is selected from the group consisting of tyloxapol, Tween 80, and phospholipids.

53. The kit of claim 37, wherein the solid composition is in the form of a single unit.

54. The kit of claim 53, wherein the solid composition is in the form of a lyophilized matrix.

55. The kit of claim 53, wherein the solid composition a compressed tablet.

56. The kit of claim 53, wherein the solid composition is film- or foil-shaped.

57. The kit of claim 37, wherein the solid composition is in the form of multiple units.

58. The kit of claim 57, wherein the solid composition comprises a lyophilized powder.

59. The kit of claim 57, wherein the solid composition is a soluble coating layer which is coated on a multiple unit carrier.

60. The kit of claim 59, wherein the multiple unit carrier is insoluble.

61. The kit of claim 59, wherein the multiple unit carrier consists of beads made from a material selected from the group consisting of glass, polymers, metals, and mineral salts.

62. The kit of claim 59, wherein the multiple unit carrier is soluble.

63. The kit of claim 59, wherein the soluble coating further comprises a binder, such as a saccharide, a sugar alcohol, or a film-forming polymer.

64. The kit of claim 37, wherein the aqueous liquid is capable of dissolving the solid composition within 30 seconds.

65. The kit of claim 37, wherein the aqueous liquid and the solid composition are formulated to form a liquid pharmaceutical composition having an osmolality from about 150 mOsmol/kg to about 600 mOsmol/kg.

66. The kit of claim 37, wherein the aqueous liquid and the solid composition are formulated to form a liquid pharmaceutical composition having a pH from about 3.5 to about 10.5.

67. The kit of claim 37, wherein the aqueous liquid and/or the solid composition comprises a buffer or a buffer salt, and wherein the aqueous liquid and the solid composition are formulated to form a liquid pharmaceutical composition having a buffer capacity _{β} from about 0.01 to about 0.7.

68. The kit of claim 37, wherein the active compound is unstable in an aqueous environment.

69. A method of treating a mammal suffering from or susceptible to a disease or condition affecting the respiratory system, comprising administering to the mammal a composition of the kit of claim 37.

70. The method of claim 69 wherein the mammal is suffering from or susceptible to bronchitis, asthma, chronic obstructive pulmonary disease, allergies, cystic fibrosis, pneumonia, bronchiectasis, bronchiolitis, lung cancer, fibrosis, pulmonary hypertension, respiratory distress syndrome, bacterial or viral infection, tuberculosis or sinusitis.

71. The method of claim 69 wherein the composition is administered through the nose and/or lungs of the mammal.

72. The kit of claim 37 wherein the active compound is selected from the group consisting of substances for diagnostic purposes such as metacholin or antiasthmatics, comprising beta-agonists, such as salbutamol, levalbuterol, formoterol, fenoterol, salmeterol, bambuterol, brocaterol, clenbuterol, terbutalin, tulobuterol, epinephrin, isoprenalin, orciprenalin, hexoprenalin; anticholinergics, such as tiotropium, oxitropium, ipratropium, glycopyrrolate; local anaesthetics, such as lidocain and derivatives thereof, mucolytics and surfactants, such as acetylcystein, ambroxol, carbocystein, tyloxapol, dipalmytoylphosphatidylcholin, recombinant surfactant proteins, D-nase; anti-inflammatory drugs comprising mediator cell inhibitors, such as cromoglycate, nedocromil, lidocaine, elastane-, leucotriene-, bradykinin-antagonists; corticosteroids, such as beclomethasone, betamethasone, budesonide, ciclesonide, flunisolide, fluticasone, icomethasone, mometasone, rofleponide, triamcinolone; bradykinine-, prostaglandine-, leucotriene- and platelet activating factor antagonists; antibiotics, including beta-lactam antibiotics, such as amoxicillin, piperacillin, clavulan acid, sulbactam; cephalosporines, e.g. cefaclor, cefazedon, Cefuroxim, Cefoxitin, cefodizim, cefsulodin, cefpodixim, cefixim; carbapenemes, such as imipenem and cilastatin; further monbactames, e.g aztrenonam; aminoglycosides, such as streptomycin, neomycin, colistin, paromomycin, kanamycin, gentamycin, amicacin, tobramycin, spectinomycine; tetracyclines, such as doxycyclin, minocycline; makrolides, such as erythromycine, clarithromycine, roxithromycine, azithromycine, josamycine, spiramycine; gyrase inhibitors or quinolones, such as ciprofloxacin, ofloxacine, levofloxacine, pefloxacine, lomefloxacine, fleroxacine, clinafloxacine, sitafloxacine, gemifloxacine, balofloxacine, trovafloxacine, gatifloxacine, moxifloxacine; sulfonamides and nitroimidazoles, including metronidazol, timidazol, chloramphenicol, lincomycine, clindamycine, fosfomycine; glycopeptides such as vancomycine, teicoplanine; peptide antibiotics, such as peptide 4; tuberculostatics, e.g. rifampicine, isoniacide, cycloserine, terizidone, ansamycine; antimycotics and antifungals, such as clotrimazol, oxiconazol, miconazol, ketoconazol, itraconazol, fluconazol; polyene antibiotics, such as amphotericine B, natamycine, nystatine, terbinafine, colistine, flucytosine; chemotherapeutics like pentamidine; immunesuppressors and immunemodulators, cytokines, dimepranol-4-acetate amideo benzoate, thymopentin, interferones, filgrastine, interleukine, azathioprine, ciclosporine, tacrolimus, sirolimus, rapamycine; drugs to treat pulmonary hypertension, such as prostacycline analogs, iloprost, remodulin, phosphodiesterase inhibitors, such as sildenafil, vardenafil, endothelian receptor antagonists, such as bosentane, tezosentane, virustatics, including podophyllotoxine, vidarabine, tromantadine, zidovudine; proteinase inhibitors, such as a-anti-trypsin; antioxidants, such as tocopherols, glutathion; pituitary hormones, hypothalamic hormones, regulatory peptides and their inhibiting agents, corticotropine, tetracosactide, choriogonandotropine, urofolitropine, urogonadotropine, saomatotropine, metergoline, desmopressine, oxytocine, argipressine, ornipressine, leuproreline, triptoreline, gonadoreline, busereline, nafareline, goselerine, somatostatine; parathyroide gland hormones, calcium metabolism regulators, dihydrotachysterole, calcitonine, clodronic acid, etidronic acid; thyroid gland therapeutics; sex hormones and their inhibiting agents, anabolics, androgens, estrogens, gestagenes, antiestrogenes; cytostatics and metastasis inhibitors, alkylants, such as nimustine, melphanlane, carmustine, lomustine, cyclophosphosphamide, ifosfamide, trofosfamide, chlorambucil, busulfane, treosulfane, prednimustine, thiotepa; antimetabolites, e.g. cytarabine, fluorouracil, methotrexate, mercaptopurine, tioguanine; alkaloids, such as vinblastine, vincristine, vindesine; antibiotics, such as alcarubicine, bleomycine, dactinomycine, daunorubicine, doxorubicine, epirubicine, idarubicine, mitomycine, plicamycine; complexes of secondary group elements (e.g. Ti, Zr, V, Nb, Ta, Mo, W, Pt) such as carboplatinum, cis-platinum and metallocene compounds such as titanocendichloride; amsacrine, dacarbazine, estramustine, etoposide, beraprost, hydroxycarbamide, mitoxanthrone, procarbazine, temiposide; anti-migraine drugs, such as proxibarbal, lisuride, methysergide, dihydroergotamine, ergotamine, clonidine, pizotifene; hypnotics, sedatives, benzodiazepines, barbiturates, cyclopyrrolones, imidazopyridines, antiepileptics, barbiturates, phenyloin, primidone, mesuximide, ethosuximide, sultiam, carbamazepin, valproic acid, vigabatrine; antiparkinson drugs, such as levodopa, carbidopa, benserazide, selegiline, bromocriptine, amantadine, tiapride; antiemetics, such as thiethylperazine, bromopride, domperidone, granisetrone, ondasetrone, tropisetrone, pyridoxine; analgesics, such as buprenorphine, fentanyl, morphine, codeine, hydromorphone, methadone, fenpipramide, fentanyl, piritramide, pentazocine, buprenorphine, nalbuphine, tilidine; drugs for narcosis, such as N-methylated barbiturates, thiobarbiturates, ketamine, etomidate, propofol, benzodiazepines, droperidol, haloperidol, alfentanyl, sulfentanyl; antirheumatism drugs including tumor necrosis factor-alfa, nonsteroidal antiinflammatory drugs; antidiabetic drugs, such as insulin, sulfonylurea derivatives, biguanids, glitizols, glucagon, diazoxid; cytokines, such as interleukines, interferones, tumor necrosis factor (TNF), colony stimulating factors (GM-CSF, G-CSF, M-CSF); proteins, e.g. epoetine, and peptides, e.g. parathyrin, somatomedin C; heparine, heparinoids, urokinases, streptokinases, ATP-ase, prostacycline, sexual stimulants, or genetic material.

73. The kit of claim 37 wherein the active compound is selected from the group consisting of substances for diagnostic purposes; beta-agonists; anticholinergics; local anaesthetics; mucolytics and surfactants; anti-inflammatory drugs; antibiotics; tetracyclines; sulfonamides; nitroimidazoles; glycopeptides; peptide antibiotics; tuberculostatics; ansamycine; antimycotics; antifungals; polyene antibiotics; chemotherapeutics; drugs to treat pulmonary hypertension; phosphodiesterase inhibitors; endothelian receptor antagonists; proteinase inhibitors; antioxidants; pituitary hormones; hypothalamic hormones; regulatory peptides and their inhibiting agents; parathyroide gland hormones; calcium metabolism regulators; thyroid gland therapeutics; sex hormones and their inhibiting agents; anabolics; androgens; estrogens; gestagenes; antiestrogenes; cytostatics; metastasis inhibitors; alkylants; antimetabolites; alkaloids; complexes of secondary group elements; metallocene compounds; anti-migraine drugs; antiepileptics; barbiturates; antiparkinson drugs; antiemetics; analgesics; drugs for narcosis; antirheumatism drugs; nonsteroidal antiinflammatory drugs; antidiabetic drugs; cytokines; proteins; peptides; or sexual stimulants.

74. The kit of claim 37 wherein the active compound is selected from the group consisting of albuterol, salbutamol, R-salbutamol, bitolterol, carbuterol, tretoquinol, formoterol, clenbuterol, reproterol, pirbuterol, tulobuterol, procaterol, bambuterol, mabuterol, tiaramide, budenoside, fluticasone, beclometasone, deflazacort, TBI-PAB, flunisolide, cloprednol, emedastine, epinastine, oxatomide, azelastine, pemirolast, repirinast, suplatast, nedocromil, oxitropium, flutropium, triamcinolone, allergy vaccines, zafirlukast, montelukast, ramatroban, seratrodast, TJ-96, ibudilast, tranilast, Iodoxamide, TO-194, pranlukast, letosteine, ketotifen, amlexanox, zileuton, Efamol Marine, tazanolast, ribavirin, pentamidine, colistin, amphotericin B, ozagrel, including their derivatives, salts, conjugates, isomers, epimers, diastereomers, or racemic mixtures.

75. The kit of claim 37 further comprising an effervescent couple consisting two reagents capable of forming a gas upon reacting with each other in an aqueous environment.

76. The kit of claim 75, wherein one of the two reagents of the effervescent couple is present in the solid composition, whereas the other one is present in the sterile aqueous liquid.
